(19) United States
(12) Patent Application Publication
(10) Pub. No.: US 2008/0227708 A1
(43) Pub. Date: Sep. 18, 2008

(54) Methods Of Attenuating Prostate Tumor Growth By Insulin-Like Growth Factor Binding Protein-3 (IGFBP-3)

(76) Inventors: Josef Silha, Winnipeg (CA); Janice G. Dodd, Winnipeg (CA); Liam J. Murphy, (US); Leigh Murphy, Cancer care Manitoba (CA)

Correspondence Address: ST. ONGE STEWARD JOHNSTON & REENS, LLC, 986 BEDFORD STREET, STAMFORD, CT, 06905-5619, US

(21) Appl. No.: 12/036,822

(22) Filed: Feb. 25, 2008

Related U.S. Application Data
(63) Continuation of application No. PCT/CA2006/001395, filed on Aug. 23, 2006.
(60) Provisional application No. 60/710,893, filed on Aug. 25, 2005.

(51) Int. Cl. A61K 38/16

(52) U.S. Cl. 514/12

(57) ABSTRACT

Insulin-like Growth Factor Binding Protein-3 (IGFBP-3) inhibits cell growth and promotes apoptosis by sequestering free Insulin-like Growth Factor (IGF), and also demonstrates IGF-independent, pro-apoptotic, anti-proliferative effects on prostate cancer cells. Prostate tumor size was significantly attenuated in transgenic mice over-expressing IGFBP-3 compared with wild-type mice. In addition, a marked reduction in late-stage tumor growth was apparent in transgenic mice over expressing mutant-IGFBP-3 indicating that the IGF-independent effects of IGFBP-3 are related to inhibiting tumor progression.

### CROSS-REFERENCE TO RELATED APPLICATIONS

The present application is a continuation of pending International patent application PCT/CA2006/001395 filed on Aug. 23, 2006 which designates the United States and claims the benefit under 35 U.S.C. §119(e) of the U.S. Provisional Patent Application Ser. No. 60/710,893, filed on Aug. 25, 2005, the content of which is incorporated herein by reference.

### FIELD OF THE INVENTION

Insulin-like Growth Factor Binding Protein-3 (IGFBP-3) inhibits cell growth and promotes apoptosis by sequestering free Insulin-like Growth Factor (IGF), and also demonstrates IGF-independent, pro-apoptotic, anti-proliferative effects on prostate cancer cells. Prostate tumor size was significantly attenuated in transgenic mice over-expressing IGFBP-3 compared with wild-type mice. In addition, a marked reduction in late-stage tumor growth was apparent in transgenic mice over expressing mutant-IGFBP-3 indicating that the IGF-independent effects of IGFBP-3 are related to inhibiting tumor progression.

### BACKGROUND OF THE INVENTION

Epidemiological studies have demonstrated that high plasma levels of IGF-I and low IGFBP-3 concentrations are associated with increased risk of prostate cancer [1, 2]. IGFBP-3 appears to both to inhibit the actions of IGF-I and -II, and also to act in an IGF-independent manner to promote apoptosis and inhibit cellular proliferation of variety of cell lines [3-5].

The N-terminal domain of IGFBP-3 appears to be important for binding to IGF-I and -II with high affinity. Mutations in the N-terminal of IGFBP-3 result in molecules that do not bind IGF-I or -II [13]. Six residues, Ile56, Tyr57, Arg75, Leu77, Leu80, Leu81 have been identified as important in high affinity binding of IGF to IGFBP-3.

Of these, Ile56, Leu80, and Leu81 are most important and substitution with glycine or alanine results in a mutant IGFBP-3 that lack the ability to bind IGF-I or IGF-II, but retain their ability to bind plasma membranes [5] and promote apoptosis and inhibit proliferation in prostate and breast cancer cell lines [5,14-15].

Under in vitro conditions it is possible to demonstrate multiple and opposing effects of IGFBP-3 on cell proliferation and apoptosis. IGFBP-3 has IGF-dependent antiproliferative, pro-apoptotic effects related to binding IGF-I and prevents access of IGF-I to the IGF-IR. Under certain conditions IGF-dependent effects of enhancing cell survival and proliferation, possibly by enhancing delivery of IGF-I to the cell membrane receptor, can also be demonstrated with IGFBP-3 [26].

The pro-apoptotic effects of IGFBP-3 are also both dependent on, and independent of p53 [9, 10]. Impaired function of the tumor suppressor protein p53 is involved in the pathogenesis of prostate cancer [11] and increased IGFBP-3 expression is an important downstream mediator of p53 action in prostate and other cancer cells [10,12]. Over expression of the large T-antigen in LPB-Tag transgenic mice inactivates p53 and results in prostate tumorigenesis.

Low plasma IGFBP-3 levels have been reported to have predictive value in identifying individuals with advanced-stage prostate cancer [2]. However, a causal relationship to explain the associations between the IGF system and prostate cancer progression in patients is lacking.

In vivo data to verify the in vitro observations regarding the effects of IGFBP-3 in cultured prostate cancer cells would be desirable. To date, the IGF-independent effects of IGFBP-3 on prostate cancer have only been demonstrated in vitro. Such results have been valuable in providing insight into the potential role of IGF and p53 in prostate tumorigenesis, however, due to the complex and numerous potential interactions of IGFBP-3 with various in vivo processes, it is unknown whether IGFBP-3 will ultimately provide any benefit or insight regarding the treatment or progression of prostate cancer in mammals.

Applicant has previously generated transgenic mice that over express human IGFBP-3 using the phosphoglycerate kinase (PGKBP-3) and cytomegalovirus (CMVBP-3) promoters [16]. These mice demonstrate fetal and post-natal growth retardation. More recently, Applicant has generated transgenic mice that overexpress the I56G/L80G/L81 GmutantIGFBP-3 (PGKmBP-3) [17]. The PGKmBP-3 transgenic mice do not have a growth-retarded phenotype.

### SUMMARY OF THE INVENTION

IGFBP-3 inhibits cell growth and promotes apoptosis by sequestering free IGFs, and also demonstrates IGF-independent, pro-apoptotic, anti-proliferative effects on prostate cancer cells. Over expression of the large T-antigen (Tag) under the rat probasin promoter in LPB-Tag mice results in prostate tumorigenesis which progresses in a manner similar to that observed in human prostate cancer. LPB-Tag mice were crossed with transgenic mice which overexpress IGFBP-3 under the cytomegalovirus promoter and the phosphoglycerate kinase promoter, CMVBP-3 and PGKBP-3 mice respectively, and also PGKmBP-3 mice that express I56G/L80G/L81G-IGFBP-3, a mutant, that does not bind IGF-I but retains IGF-independent pro-apoptotic effects in vitro. Prostate tumor size and expression of p53 was significantly attenuated in LPB-Tag/CMVBP-3 and LPB-Tag/PGKBP-3 mice compared with LPB-Tag/Wt mice. A more marked effect was observed in LPBTag/CMVBP-3 compared with LPB-Tag/PGKBP-3 reflecting increased levels of transgene expression in CMVBP-3 prostate tissue. Similar elevated levels of serum IGFBP-3 were apparent in CMVBP-3 and PGKBP-3 mice emphasizing the importance of local rather than circulating IGFBP-3 in the attenuation of prostate tumorigenesis. No attenuation of tumor growth was observed in LPB-Tag/PGKmBP-3 mice during the early tumor development indicating that the inhibitory effects of IGFBP-3 were most likely IGF-dependent during the initiation of tumorigenesis and early growth. At 15 weeks of age expression of dorsolateral proteins, a marker of differentiated prostate function was lost in LPB-Tag/Wt and LPB-Tag/PGKmBP-3 tissue but preserved in LPB-Tag/PGKBP33 tissue. In contrast epidermal growth factor receptor (EGF-R) expression was increased in LPB-Tag/Wt and LPB-Tag/PGKmBP-3 tissue compared to LPB-Tag/PGKBP-3. IGF receptor was similarly increased in all transgenic mice compared to Wt mice but pAkt expression a marker of downstream IGF-I action was increased in LPB-Tag/Wt and LPBTag/PGKmBP-3 but not in LPB-Tag/PGKBP-3 or LPB-Tag/CMVBP-3 mice. After 15 weeks of age a marked reduction in tumor growth was apparent in LPB-Tag/PGKmBP-3 mice compared to LPB-Tag/Wt mice indicating that the IGF-independent effects of IGFBP-3 may be important in inhibiting tumor progression.

In accordance with the invention, there is provided a method of reducing prostate cancer tumorigenesis in vivo comprising introducing an effective amount of insulin growth factor binding protein-3 (IGFBP-3) into prostate cancer cells. The prostate cancer may be early-stage or late stage cancer and the IGFBP-1 may be mutant IGFBP-1.

### BRIEF DESCRIPTION OF THE DRAWINGS

FIG. 1. IGFBP-3 over expression attenuates prostate tumor development. Prostate weight was assessed in the various mice at different ages. The data represent the mean +SEM. The number of mice killed at each time point is shown above. For simplicity, only a single line has been used to depict data for Wt/Wt, PGKBP-3/Wt and CMVBP-3/Wt mice that did not differ significantly from each other. * and ** indicates p<0.05 and p<0.01 respectively, for the difference between the double transgenic mice and LPB-Tag/Wt mice as determined by ANOVA and Tukey HSD test.

FIG. 2. Serum IGF-I and human IGFBP-3 levels and prostate transgene-derived mRNA levels in PGKBP-3/Wt and CMVBP-3 mice. In panel A, the data represent the mean ±SEM levels for N=5 or more mice per group at ˜4 months of age. * and ** indicates p<0.01 and p<0.001 respectively, for the difference between the transgenic and wild-type mice. Panel B represents an RNase protection assay using a human IGFBP-3 specific probe. Cyclophilin is included as an internal control.

FIG. 3. Mutant IGFBP-3 overexpression attenuates prostate tumor development at the later time points. Prostate weight was assessed in the various strains of mice at different ages. The data represent the mean +SEM. The number of mice killed at each time point is shown above. For simplicity only a single line has been used to depict data for Wt/Wt, PGKBP-3/Wt and PGKmBP-3/Wt mice that did not differ significantly from each other. ** indicates p <0.01 for the difference between the prostate weight in LPBTag/PGKmBP-3 and LPB-Tag/PGKBP-3 mice as determined by ANOVA followed by the Tukey HSD test. N. S indicates no significant difference between LPB-Tag/PGKmBP-3 and LPB-Tag/PGKBP-3 mice. # indicates p<0.001 for the difference between LBPTag/PGKmBP-3 and LPB-Tag/Wt for the data from 17, 19 and 21 weeks combined.

FIG. 4. Expression of IGFBP-3 in prostate tumors. Panel A shows an immunoblot of prostate extracts from various mouse strains at 15 weeks of age using a human IGFBP-3 specific antibody. Panel B shows a Western ligand blot using 125I-IGF-I of the same gel. Panel C depicts a Western ligand blot of prostate tissue extracts at 21 weeks of age.

FIG. 5. Expression of p53 and loss of expression of dorsolateral protein in prostate tumors. Prostate extracts from 15 week old mice were analyzed by immunoblotting with anti-human IGFBP-3 (Panel A). The same filter was subsequently reprobed with anti-p53 antibody (Panel B). In Panel C a separate filter was probed with antibody against dorsolateral protein.

FIG. 6. Expression of EGF and IGF-I receptor and phospho-Akt(Ser473) in prostate tumors. Immunoblots were quantified by densitometry. Data represent two or more samples for each group. * indicates p<0.05 for the difference between transgenic and wild-type mice.

### DETAILED DESCRIPTION OF THE INVENTION

### Materials and Methods

### Transgenic Mice

The generation and characterization of PGKBP-3, CMVBP-3 and PGKmBP-3 transgenic mice have been previously reported [16, 17]. The I56G/L80G/L81G-mutant IGFBP-3 plasmid was generated by site-directed mutagenesis and sub-cloned downstream of the phosphoglycerate promoter in the same plasmid used for the generation of PGKBP-3 mice [17].

The 12T-5 strain of LBP-Tag transgenic mice that express the SV-40 large T antigen under the prostate specific probasin promoter was used for these studies [18]. The 12T-5 strain of LBP-Tag mice develop palpable prostate tumors starting at approximately 2 months of age. All transgenic mice were generated in the same CD-1 genetic background. Homozygous male PGKBP-3, CMVBP-3 or PGKmBP-3 mice and normal wild-type male CD-1 mice were bred with heterozygous LBP-Tag female mice. Male F1 offspring were genotyped and approximately 25% of all the offspring were double transgenic male animals. These were killed at various ages for determination of prostate size and histology. The presence of the various transgenes was detected either by Southern blot analysis or by PCR using tail DNA as previously described [16-18].

### IGFBP-3 and IGF-I Assays

Human IGFBP-3 was measured using an immunoradiometric assay from Diagnostic Systems Laboratories (Webster, Tex.). Total plasma IGF-I was measured by a sensitive rat IGF-I radioimmunoassay using an assay kit (Linco Research Inc., St. Charles, Mo., USA).

RNA extraction and RNase Protection Assays

Total RNA was extracted from prostate tissue and tumors using TRizol reagent (Invitrogen). The concentration of RNA was determined spectrophotometrically and the integrity of the RNA in all samples was documented by visualization of the 18 and 28S ribosomal bands after electrophoresis through a 0.8% formaldehyde/agarose gel. Maxiscript SP6/T7 and RPAIII kits (Ambion, Austin Tex.) were used for the RNase protection assay.

A 267-bp fragment containing the sequence corresponding to the 3′-end of the human IGFBP-3 cDNA and the bovine GH polyadenylation signal was used as a template as previously described [16]. A mouse cyclophilin riboprobe was used as the internal standard and century RNA markers from Ambion were used to determine the size of the protected fragment. The protected sizes for the transgene-derived RNA and cyclophilin fragments were 267 and 103 bp, respectively.

### Immunoblotting

Prostate tissue sample, ˜20 μg of protein, were mixed with 10 μl of loading buffer and heated in boiling water for 5 min. The samples were separated on 10% SDS PAGE, and proteins were transferred to nitrocellulose membranes. Membranes were blocked in 5% nonfat milk, incubated with a 1:500 dilution of rabbit polyclonal anti-human IGFBP-3 (Santa Cruz Biotechnology) antibody for 2 h at RT. After incubation, membranes were washed three times (5 min each) in TBST (10 mM Tris, 150 mM NaCl, 0.05% Tween 20, pH 8.0) and incubated with a 1:5000 dilution of anti-rabbit horseradish peroxidase-conjugate (Santa Cruz Biotechnology, Santa Cruz, Calif.) for 1 h at RT.

After washing (3×5 min) in TBST, membranes were analyzed with ECL detection system. For p53 and epidermal growth factor receptor (EGF-R), rabbit polyclonal antibodies from Santa Cruz Biotechnology were used at a 1:400 dilution. Rabbit polyclonal antibody against the C-terminus of the beta chain of the IGF-I receptor from Santa Cruz Biotechnology was used at a dilution of 1:1000. Rabbit polyclonal antibody phospho-AKT (Ser473) was obtained from Cell Signaling (Beverly, Mass.), and used at a dilution of 1:1000. Rabbit antibody against dorso lateral proteins was a gift from Dr. G. Cunha (UCSF) and was used at a dilution of 1:40,000. This antibody recognizes androgen dependent dorsolateral prostate secreted proteins and is a marker of differentiate prostate function [19].

### Ligand Blotting

For ligand blotting 20 μg of protein samples (without DTT) were separated by SDS-PAGE on a 10% gel and transferred to the nitrocellulose membrane as mentioned above. After blocking with 5% nonfat milk, membranes were incubated in 10 mM Tris-HCl buffer (pH 7.4), 150 mM NaCl, 3% Nonidet P-40 containing 400,000 cpm 125I-IGF-I for 3 h at RT. After washing (3×15 min each) with the same buffer without radio-ligand, the membranes were exposed to Kodak MR film at −70° C.

### Statistical Analysis

All data are expressed as the mean ±SEM. Statistical analysis was initially performed using an analysis of variance (ANOVA) and the Tukey HSD test using online statistical software (http://faculty.vassar.edu/lowry/VassarStats.html). Comparison was made between LPB-Tag/Wt mice and the other groups of mice both for the whole data set and for data from each time point. Prostate weight was log transform and least squares regression analysis was used to determine the lines of best fit and their confidence limits. The statistical significance of the difference in the slope and intercept was then determined.

### Results

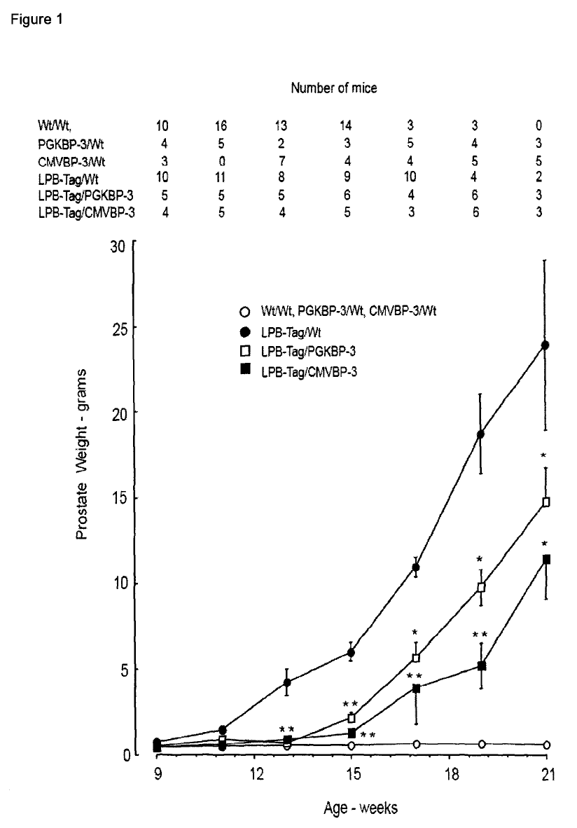

The increase in prostate weight with age in Wt/Wt, LPB-Tag/Wt, LPBTag/CMVBP-3, and LPB-Tag/PGKBP-3 mice is shown in FIG. 1. There was no significant difference in prostate weights in Wt/Wt, CMVBP-3/Wt and PGKBP-3/Wt mice. For the purpose of clarity only a single curve is shown for Wt/Wt, CMVBP-3/Wt and PGKBP-3/Wt mice in FIG. 1.

Prostate tumorigenesis, as assessed by prostate weight, was markedly attenuated by overexpression of IGFBP-3 under either the CMV or PGK promoters (p<0.001 by ANOVA). This attenuation was more marked in LPBTag/CMVBP-3 mice compared to LPB-Tag/PGKBP-3 mice (p<0.05). Once initiated, prostate tumors grew at a slightly slower rate in LPB-Tag/CMVBP-3, and LPBTag/PGKBP-3 mice than LPB-Tag/Wt mice (FIG. 1). Prostate tumor weight was log transformed and least squares regression analysis was used to obtain the line of best fit for the relationship between prostate weight and time. The slope of this relationship was significantly less in LPBTag/CMVBP-3 compared with LPB-Tag/Wt mice (p<0.001) and similar trend was apparent in LPB-Tag/PGKBP-3 mice (Table 1).

TABLE 1
Regression analysis of prostate weight versus age in transgenic mouse strains.

| | LPB-Tag/Wt | LPB-Tag/PGKBP-3 | LPB-Tag/CMVBP-3 |
|---|---|---|---|
| Slope | 0.131 ± 0.004 | 0.126 ± 0.006 | 0.099 ± 0.007** |
| Y-intercept | −1.269 ± 0.061 | −1.499 ± 0.082* | −1.274 ± 0.099 |
| Correlation | R = 0.971 | R = 0.945 | R = 0.896 |
| Coefficient | | | |
| Doubling time | 2.29 ± 0.07 | 2.39 ± 0.12 | 3.07 ± 0.22* |
| at 10 g - weeks | | | |

*p < 0.05 and
**p < 0.001 for the difference between double Transgenic and LPB-Tag/Wt mice

In LPB-Tag/Wt mice a prostate weight of 10 grams was achieved at ˜17 weeks of age. This weight was achieved after a further delay of ˜2.5 and ˜5 weeks in LPB-Tag/PGKBP-3 and LPBTag/CMVBP-3 mice respectively. Although the PGKBP-3 and CMVBP-3 mice were approximately 10% smaller than Wt mice [16], differences in body weight did not account for the apparent reduction in prostate tumor growth. A significant reduction in relative weight of the prostate gland was still apparent when expressed as a percentage of total body weight. Examination of the different lobes of the prostate gland in various transgenic strains gave similar results to that seen when the whole prostate gland was considered (data not shown).

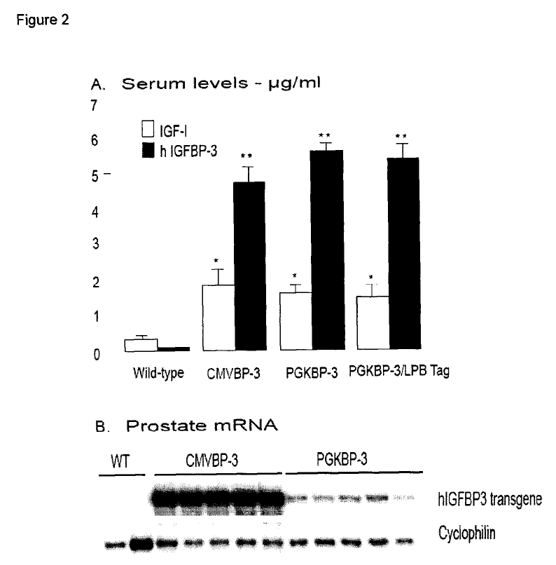

In an attempt to understand the differences in prostate tumor growth in LPBTag/CMVBP-3 and LPB-Tag/PGKBP-3 mice, we examined plasma levels of IGF-I, human IGFBP-3 transgene and the abundance of the transgene-derived mRNA in prostate tissue from CMVBP-3 and PGKBP-3 mice. Plasma levels of the transgene-derived IGFBP-3 were similar in CMVBP-3/Wt and PGKBP-3/Wt mice and were also similar to that seen in LPB-Tag/PGKBP-3 (FIG. 2A). The same was true for IGF-I, which was significantly increased in CMVBP-3/Wt, PGKBP-3/Wt and LPB-Tag/PGKBP-3 mice compared to Wt/Wt control mice of similar age, reflecting the increased IGF-I binding capacity in the serum.

However, transgene expression was markedly increased in prostate tissue from CMVBP-3/Wt compared to PGKBP-3/Wt mice (FIG. 2B). The RNase protection assay is specific for the transgene and does not detected murine IGFBP-3 [17], hence the absence of signal in the lanes containing prostate RNA from Wt/Wt mice. The abundance of hIGFBP-3 mRNA in prostate tissue from CMVBP-3/Wt mice was increased 5.6+0.9 fold compared to PGKBP-3/Wt mice, p<0.001.

The phenotypic manifestations of over expression of mutant IGFBP-3 in PGKmBP-3 mice have been previously reported [17]. These mice do not demonstrate growth retardation and have slightly higher levels of IGF-I and murine IGFBP-3 than Wt mice possibly reflecting compensation for the IGF-independent growth inhibiting effects of mutant IGFBP-3 [17].

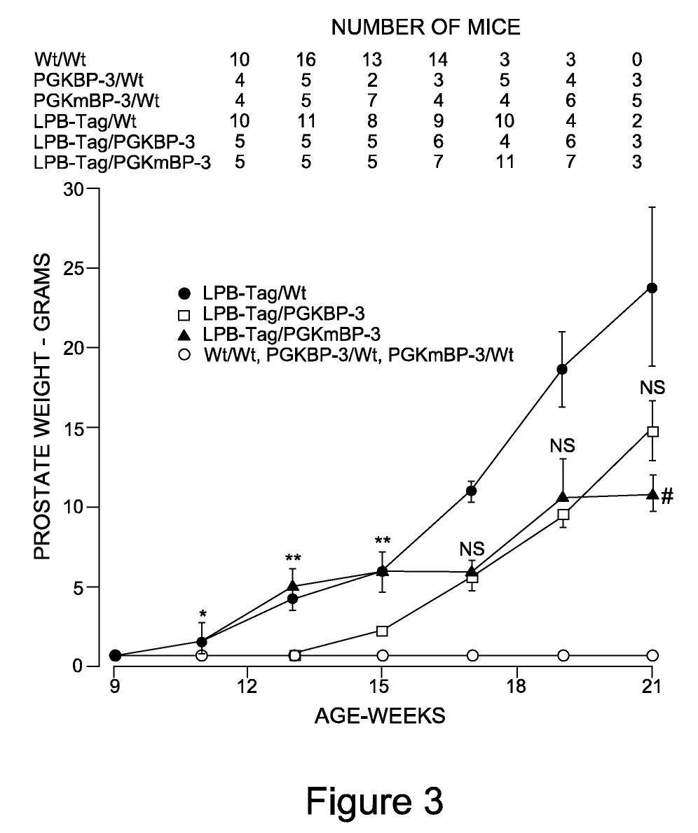

There was no significant difference in prostate tumor growth in LPB-Tag/Wt and LPB-Tag/PGKmBP-3 mice for the first 15 weeks of life (FIG. 3).

However, a marked reduction in tumor growth was observed in LPB-Tag/PGKmBP-3 mice after 15 weeks of age and at subsequent time points there was no significant difference in prostate tumor size in LPB-Tag/PGKmBP-3 and double transgenic mice expressing the intact IGFBP-3 driven by the same promoter.

A total of 43 LPBTag/PGKmBP-3 mice were examined from 3 different PGKmBP-3 male stud mice. Since the different stud males contributed different numbers of offspring to the different time points it was investigated whether there was any differences in prostate weight in offspring of different male studs at 15 and 17 weeks where there was adequate representation of offspring from all three male studs. When prostate weight for individual mice was expressed as a percentage of mean prostate weight for the whole group at each time point, there was no significant difference in prostate weight of the offspring of different PGKmBP-3 stud males compared to the mean for the whole group.

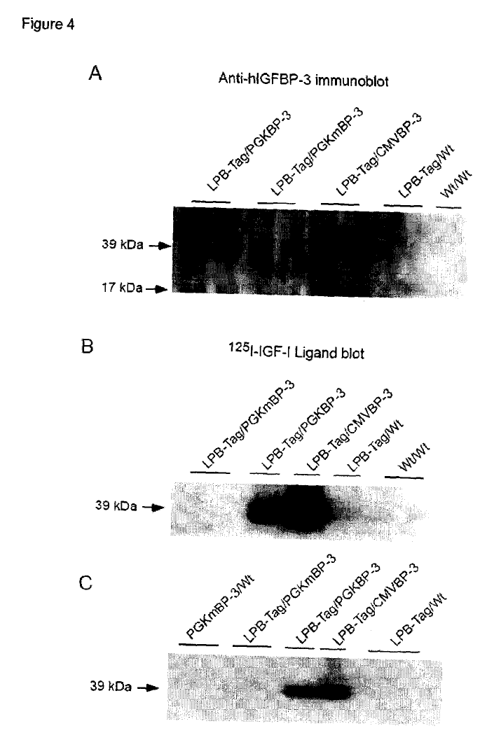

Immunoblotting and Western ligand blotting was used to investigate the presence of the transgene-derived protein product in prostate tissue from the various transgenic strains. Using antibody specific for hIGFBP-3, an intense signal was apparent in lanes containing prostate extract from LPB-Tag/CMVBP-3 mice (FIG. 4a).

Both intact hIGFBP-3 of ˜40 kDa and a less abundant ˜19 kDa IGFBP-3 proteolytic fragment, previously reported in other tissue extracts [20], was apparent. hIGFBP-3 was also detected, but less abundant, in prostate extracts from LPB-Tag/PGKBP-3 mice.

No signal was observed in extracts from LPB-Tag/Wt or Wt/Wt mice.

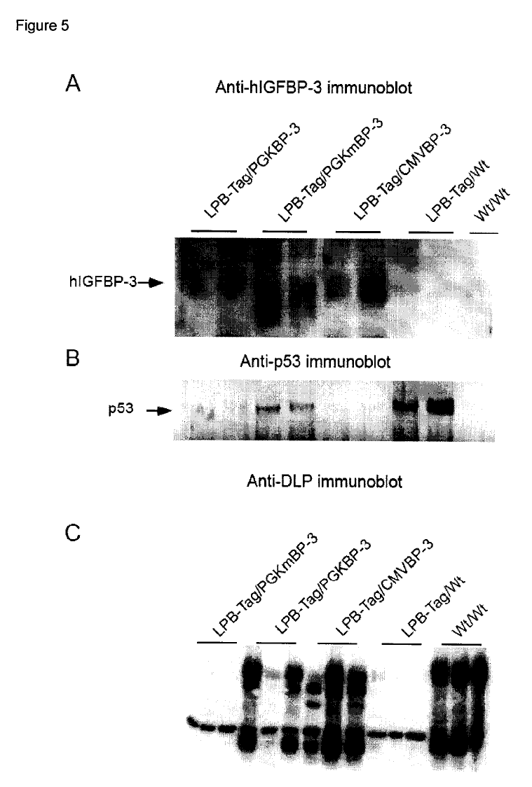

Weak immunoreactivity was apparent in lanes containing extracts from LPB Tag/PGKmBP-3 mice (FIG. 4a and FIG. 5a). In extracts from these mice, the ˜40 kDa hIGFBP-3 immunoreactivity was present as a smear and the ˜19 kDa fragment was not seen suggesting the possibility of extensive degradation of the non-IGF binding mutant IGFBP-3.

Western ligand blotting with ¹²⁵I-IGF-I confirmed the higher level of expression of the transgene in prostate tissue of LPB-Tag/CMVBP-3 mice compared to LPBTag/PGKBP-3 mice (FIG. 4b).

No binding was observed in lanes containing prostate extract from LPB-Tag/PGKmBP-3 mice since this mutant IGFBP-3 does not bind IGF-I [17].

Radioactivity was also not detected in lanes containing extracts from LPB-Tag/Wt and Wt/Wt mice probably because of the low sensitivity of this technique in detecting endogenous murine IGFBPs in prostate tissue extracts under these conditions.

The data shown in FIG. 4b are from prostate tissue obtained at 15 weeks of age. To exclude a possible mix up of LPB-Tag/PGKBP-3 and LPB-Tag/PGKmBP-3 at the later time points Western ligand blotting was used to analyze samples collected at 21 weeks of age.

Similar results were obtained with these tissues (FIG. 4c). 125I-IGF-I binding was observed in lanes containing extracts from LPB-Tag/CMVBP-3 and LPB-Tag/PGKBP-3 mice but not from LPB-Tag/PGKmBP-3 mice. The difference in signal intensity between FIGS. 4b and 4c results from differences in decay in radiolabel and autoradiography exposure time and no meaningful conclusions can be drawn concerning the abundance of transgene expression at the two time points.

The presence of immunoreactive p53 was assessed in prostate tissue in various mouse strains at 15 weeks of age. In LPB-Tag/Wt mice the SV40 large T-antigen stabilizes p53.

Immunoreactivity was detected in prostate extracts from these mice (FIG. 5). A lower level of p53 protein was also detected in extracts from LPBTag/PGKmBP-3 mice. No p53 was apparent in lanes containing extracts from Wt/Wt, LPB-Tag/PGKBP-3 or LPB-Tag/CMVBP-3 mice. Expression of dorsolateral proteins, a marker of differentiated function [19], was lost in prostate tissue from LPB-Tag/Wt and LPB-Tag/PGKmBP-3 mice but relatively preserved in LPB-Tag/CMVBP-3 and LPBTag/PGKBP-3 mice (FIG. 5c).

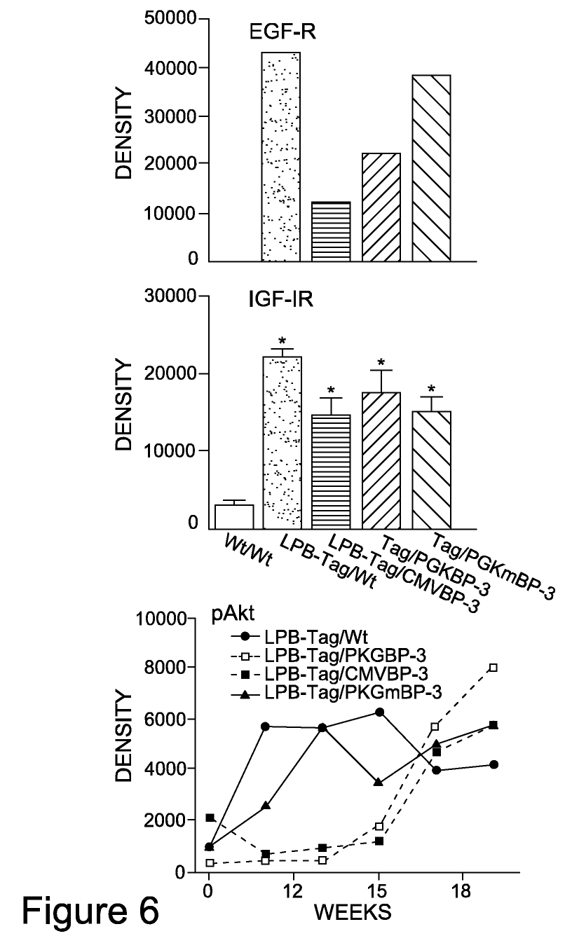

Expression of both EGF-R and IGF-IR were unregulated in the prostate tumors (FIG. 6). EGF-R was most abundant in LPB-Tag/Wt and LPB-Tag/PGKmBP-3 mice and the lowest levels of expression were apparent in tissue from LPB-Tag/CMVBP-3 mice.

In contrast IGF-IR was significantly elevated in all transgenic mice carry the LPB-Tag transgene compared to Wt mice and there was no significant difference between those expressing intact or mutant IGFBP-3. Despite increased levels of IGF-IR in LPBTag/CMVBP-3 and LPB-Tag/PGKBP-3 mice, the abundance of phospho pAkt(Ser 473) was reduced in these mice compared to LPB-Tag/Wt and LPB-Tag/PGKmBP-3 mice (FIG. 6, lower panel) suggesting that signaling at the IGF-IR was attenuated in the double transgenic mice expressing intake IGFBP-3.

### Discussion

Heterozygous LBP-Tag mice carry a genetic predisposition to neoplasia restricted to the prostate because of the tissue specificity of the long probasin promoter [18].

The SV40 large T oncoprotein interferes with cellular tumor suppressor proteins such as p53, mimicking molecular alterations that occur in human prostate cancer [11,18]. The LPBTag mice have the distinct advantage over other transgenic models of prostate cancer in that it faithfully reproduces the sequence of progression seen in human prostate cancer. In particular the histological feature of the putative precursor lesions, prostatic intraepithelial neoplasia (PIN) is also apparent in this mouse model. In addition biomarkers associated with human PIN that predict progression to invasive carcinoma are also evident in the mouse model including, increased PCNA levels a marker of proliferation, decreased apoptosis, enhanced growth factor receptor expression (erbB family), elevated nm23, PTEN and c-met oncogene expression, and increased expression and nuclear localization of the androgen receptor.

The marked reduction of tumor growth seen in both LPB-Tag/PGKBP-3 and LPBTag/CMVBP-3 mice was predominantly due to paracrine/autocrine effects in the prostate rather than the result of systemic IGFBP-3 since the effect was more marked in LPBTag/CMVBP-3 mice that have higher levels of transgene expression in the prostate but similar levels of circulating IGFBP-3 to LPB-Tag/PGKBP-3 mice.

Furthermore, attenuation of prostate tumorigenesis was apparent despite significantly increased levels of IGF-I in the circulation in both these strains of double transgenic mice [16].

After 15 weeks of age the tumors in LPB-Tag/PGKBP-3 and LPB-Tag/CMVBP-3 grew rapidly, although at a slightly slower rate than that seen in LPB-Tag/Wt mice suggesting that the predominant effect of over expression of IGFBP-3 in these mice was at the early stages of tumor development. The mechanisms involved in tumor development in LPBTag mice are not fully understood but tumor development is delayed until after sexual maturation in this model and is clearly androgen dependent [18]. Both PGKBP-3 and CMVBP-3 male mice are fertile and testosterone levels are not markedly different in these mice and Wt mice [16]. The observations in LPB-Tag/PGKBP-3 and LPBTag/CMVBP-3 mice suggest that prostate cancer development is IGF-I dependent in the early stages whereas the tumor progression may be less dependent on IGF-I as the disease progresses. In these mice, tumor development was delayed due to IGF-dependent action of IGFBP-3 but, once well established, the tumor appeared to grow at a rate approaching that seen in LPB-Tag/Wt mice.

Prostate tumor development and growth in LPB-Tag/PGKmBP-3 was similar to LPB-Tag/Wt mice during the first 15 weeks. Since mutant IGFBP-3 does not bind IGF-I [17], it would be unable to inhibit IGF-I action during the critical early stages of prostate tumorigenesis. It has been previously shown that PGKmBP-3 transgenic mice have low levels of human IGFBP-3 in the circulation (˜0.5 μg/ml) compared to PGKBP-3 transgenic mice, (˜5 μg/ml), despite identical transgene promoters and similar levels of tissue transgene mRNA [16, 17].

Applicant believes that the mutant IGFBP-3 was more rapidly cleared from the circulation and degraded, since mutant IGFBP-3 is unable to bind IGF-I which appears to be necessary for the formation of stable ternary complexes with the acid-labile subunit [24].

Western blotting of prostate extracts confirmed that mutant IGFBP-3 was more degraded than native IGFBP-3. Human prostate tissue contains prostate specific antigen that can proteolyses IGFBP-3 [25]. It is likely that mouse prostate tissue contains similar kallikreins that can degrade IGFBP-3.

The most unexpected finding was the decline in tumor growth in LPBTag/PGKmBP-3 after 15 weeks of age. This represents an IGF independent effect of IGFBP-3. Although it is unclear why these IGF-independent effects are not manifested earlier, it suggests that IGFBP-3 treatment may be beneficial in reducing tumor growth at various stages of through separate mechanisms.

Applicant carefully excluded the possibility of a mix-up of LPB-Tag/PGKmBP-3 and LPB-Tag/PGKBP-3 or LPB-Tag/CMVBP-3 mice by reviewing the parentage of each of the mice and also by analyzing the tumor extracts from 21 week old mice by Western ligand blotting (FIG. 4). Furthermore, the LPB-Tag/PGKmBP-3 offspring used for the age 19 and 21-week data points were from the same stud PGKmBP-3 male that contributed offspring to earlier time points and thus the data from the later time points is not the result of a specific stud male.

As mentioned above, IGF independent anti-proliferative, pro-apoptotic effects have been reported in vitro. This study represents the first demonstration of the IGF-independent effects of IGFBP-3 in vivo. These IGF-independent effects of IGFBP-3 demonstrated in vitro are only apparent under conditions where IGF-I is absent [5,10,14], or in cell lines which are not dependent upon IGF-I for growth because they lack IGF-I receptor [3,4]. It is possible that the IGF-independent effects of IGFBP-3 are inhibited by IGF-I or not apparent in cells where the IGF-I signal transduction pathway is activated. Thus, early in prostate tumorigenesis in LPB-Tag/PGKmBP-3 mice where the tumors are growing in response to IGF-I, these IGF-independent effects of IGFBP-3 may be blocked or masked by IGF-I stimulated mitogenesis.

An alternative explanation for the apparent lack of effect of mutant IGFBP-3 during early prostate cancer growth in this model may be related to the enhanced degradation of mutant IGFBP-3 in prostate tissue. With the loss of markers of differentiation, such as dorsolateral protein as the tumor progress, there may also be a loss of IGFBP-3 protease activity and consequently enhanced levels of mutant IGFBP-3 that could exert a progressively more marked effect with increasing tumor mass.

While the exact mechanism whereby over expression of mutant IGFBP-3 exerts its anti-proliferative effect requires further investigation, the data clearly demonstrates that local over expression of IGFBP-3 attenuates prostate tumorigenesis in early and later stage prostate tumor development. Applicant's observations support an important role of local IGF-I levels in prostate tumor progression. Furthermore, Applicant's data also suggest that the use of IGFBP-3 and its mutant may be a useful therapeutic strategy in the treatment of prostate cancer.

What is claimed is:

1. A method of reducing prostate cancer tumorigenesis in vivo comprising introducing an effective amount of insulin growth factor binding protein-3 (IGFBP-3) into prostate cancer cells.

2. A method as in claim 1 wherein the prostate cancer is early-stage.

3. A method as in claim 1 wherein the prostate cancer is late-stage.

4. A method as in claim 1 wherein the IGFBP-1 is mutant IGFBP-1.
